(19) United States
(12) Patent Application Publication
(10) Pub. No.: US 2024/0277998 A1
(43) Pub. Date: Aug. 22, 2024

(54) ROTOR BEARING SYSTEM

(71) Applicant: KARDION GMBH, Stuttgart (DE)

(72) Inventors: Andreas Vogt, Renningen (DE); Ingo Stotz, Ditzingen (DE); Johannes Bette, Leonberg (DE); Armin Schuelke, Aidlingen (DE); Xiang Li, Stuttgart (DE); Uwe Vollmer, Sindelfingen (DE); David Minzenmay, Stuttgart (DE)

(21) Appl. No.: 18/654,385

(22) Filed: May 3, 2024

Related U.S. Application Data
(63) Continuation of application No. 17/054,884, filed on Jul. 13, 2021, now Pat. No. 12,005,248.

(30) Foreign Application Priority Data
May 16, 2018 (DE) 10 2018 207 611.1

(51) Int. Cl. A61M 60/82; A61M 60/178; A61M 60/216; A61M 60/419; A61M 60/824; A61M 60/825; F04D 3/00; F04D 29/048; F04D 29/18; F16C 17/10; H02K 7/08; H02K 16/00

(52) U.S. Cl. CPC A61M 60/82; A61M 60/178; A61M 60/216; A61M 60/419; A61M 60/824; A61M 60/825; F04D 3/00; F04D 29/048; F04D 29/181; F16C 17/10; H02K 7/088; H02K 16/005; F16C 2316/18

(57) ABSTRACT

The invention relates to a rotor bearing system (1). Said system comprises a housing (80) in which a first permanent magnet (30) is mounted such that it can rotate about a first axis (105). A rotor (70) for conveying a liquid comprises a second hollow-cylindrical permanent magnet (40), which is mounted such that it can rotate about a second axis. The first permanent magnet (30) and the second permanent magnet (40) overlap axially at least partially, wherein the first permanent magnet (30) is disposed offset relative to the second permanent magnet (40). In the axial overlap region (160) of the first permanent magnet (30) and the second permanent magnet (40), the housing (80) is positioned between the two permanent magnets (30, 40). A first bearing (20) is configured for the relative axial positioning of the rotor (70) and the housing (80) with respect to one another and for receiving an axial force resulting from the arrangement of the first permanent magnet (30) and the second permanent magnet (40), and a second bearing (10) and a third bearing (90) are configured for receiving radial forces and for positioning the axis of rotation of the second permanent magnet (40).

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a continuation of U.S. patent application Ser. No. 17/054,884, entitled ROTOR BEARING SYSTEM, and filed Jul. 13, 2021, which is the U.S. National Phase of international application number PCT/EP2019/062746, entitled ROTOR BEARING SYSTEM, and filed May 16, 2019, which claims the benefit of priority to German patent application number 102018207611.1, entitled ROTOR BEARING SYSTEM, and filed May 16, 2018, the disclosure of which is hereby incorporated by reference herein in its entirety for all purposes and forms a part of this specification.

### BACKGROUND

### Field

The present invention relates to a rotor bearing system.

### Description of the Related Art

A combination of a solid-body bearing and a passive magnetic axial coupling is known in the state of the art. A radial passive magnetic coupling for torque transmission, which has an axial offset of a coupling part, i.e. a preload, for adjusting the axial force, is known as well.

The use of an axial passive magnetic coupling leads to a high axial force on the bearing arrangement, and consequently to increased friction and increased wear. The axial force to be received by the bearing cannot be minimized, because said force is a direct function of the torque to be transmitted.

Further known is also an arrangement having two fixed bearings for axial and radial support of the rotor in pumps, in particular in pumps for cardiovascular support (VAD). In the case of preloaded bearings, the use of two solid-body bearings represents an overdetermined bearing arrangement in which the preload is reduced by wear of the loaded bearing or bearings until there is a small amount of play and the bearing arrangement becomes underdetermined, which is disadvantageous. A thermally induced elongation or expansion of the rotor can furthermore not be compensated, so that the rotor may jam between the two axial bearings.

### SUMMARY

The object of the invention is to provide a rotor bearing system in which less friction and therefore less wear occurs.

This object is achieved by the rotor bearing system specified in claim 1. Advantageous embodiments of the invention are specified in the dependent claims.

A rotor bearing system according to the invention is in particular used for the contactless transmission of a torque to a rotating rotor in combination with the support thereof in axial and radial direction.

A rotor bearing system according to the invention comprises a housing, in which a first in particular cylindrical permanent magnet is mounted such that it can rotate about a first axis. A rotor bearing system according to the invention further comprises a rotor comprising a second hollow-cylindrical permanent magnet, which is mounted such that it can rotate about a second axis. The rotor preferably comprises a hollow-cylindrical part in which the second hollow-cylindrical permanent magnet is disposed.

In a rotor bearing system according to the invention, the first permanent magnet and the second permanent magnet overlap axially at least partially, wherein the first permanent magnet is disposed offset, in particular axially, relative to the second permanent magnet.

The phrase “the first permanent magnet is disposed axially offset relative to the second permanent magnet” means that an axial center of the first permanent magnet is disposed axially offset relative to an axial center of the second permanent magnet.

The axial center of a permanent magnet is hereby calculated as the point between the one axial end of the permanent magnet and the opposite other axial end of the permanent magnet. One axial end is located on an axial longitudinal axis of the permanent magnet.

The housing between the two permanent magnets is furthermore located in the axial overlap region of the first permanent magnet and the second permanent magnet.

The rotor bearing system also comprises a first bearing for the relative axial positioning of the rotor and the housing with respect to one another and for receiving an axial force resulting from the arrangement of the first permanent magnet and the second permanent magnet.

The rotor bearing system further comprises a second bearing and a third bearing for receiving radial forces and for positioning the axis of rotation of the second permanent magnet.

The axial offset of the first and the second permanent magnet relative to one another produces a force in axial direction between these bodies which, depending on the type of magnetization, acts in or preferably counter to the offset direction. This in particular makes it possible to set a positive, negative or disappearing axial force resulting from the coupling and other operating forces, e.g. from flow forces, in a defined manner, without simultaneously significantly reducing the transmittable torque. For this purpose, the axial force exerted on the rotor from the flow can be determined first, e.g. by evaluating flow simulations. The relationship between axial offset and magnetic axial force can be determined from magnetic simulations and/or measurements. An offset can then be selected for the design, in which the magnetic axial force at least compensates the flow axial force, preferably overcompensates the flow axial force by a safety factor.

In combination with the first bearing, i.e. an axial bearing, a defined axial force required for the bearing function can thus be set via the relative axial offset. Said axial force can be selected such that the first bearing can be operated within the allowable range with regard to friction and wear. It is preferred that the axial bearing is operated in constant contact, i.e. the magnetic axial force must at least compensate the flow axial force at all times.

In contrast to a solution realized in the state of the art with two axial solid-body bearings having the above-described disadvantages, the solution described here comprises only one axial solid-body bearing and thus avoids overdetermination or underdetermination. The specifically adjustable axial force from the magnetic coupling also ensures that the axial support by the solid-body bearing is sufficient in only one direction.

According to a preferred embodiment, the first permanent magnet and the second permanent magnet are arranged coaxially. This advantageously enables efficient coupling of the first and second permanent magnet.

It is further preferred that an axis of rotation of the rotor and an axis of the second permanent magnet are coaxial. This advantageously ensures that the rotor and the second permanent magnet are disposed in a symmetrical manner, which also facilitates the production of the rotor.

It is further preferred that an axis of rotation of the shaft and an axis of the first permanent magnet are coaxial. This advantageously ensures that the shaft and the first permanent magnet are easy to produce.

It is further preferred that an axis of rotation of the shaft and an axis of rotation of the rotor are coaxial. This advantageously ensures that the coupling between the first and the permanent magnet is efficient.

According to a preferred embodiment, the rotor comprises a conical or tapered part, which adjoins the hollow-cylindrical part. It is preferred here that the axis of the cone and the axis of rotation of the rotor, which is preferably coaxial with the axis of the second permanent magnet, are coaxial. The base of the conical part adjoins the hollow-cylindrical part in the direction of the bearing mounted between the rotor and the housing. The outer circumference of the base of the cone is hereby connected to the annular opening at an axial end of the hollow-cylindrical part.

According to another preferred embodiment, the second bearing is mounted on the end of the rotor facing away from the housing, i.e. on the end of the conical part facing away from the housing. It is preferred that the bearing is mounted between the rotor and an affixed component, whereby the affixed component is preferably permanently connected to the housing.

According to another preferred embodiment, the rotor comprises blades on the outer periphery of the rotor, preferably on the conical part of the rotor, which, when the rotor rotates, transports a liquid from the end of the rotor facing away from the housing toward the housing. It is preferred that the rotor comprises holes, so that the liquid is drawn from outside the rotor into a gap formed by the inner side of the hollow-cylindrical part of the rotor and an outer side of the housing, to then flow from the inside of the hollow-cylindrical part of the rotor flow through the conical part of the rotor to the end of the rotor facing away from the housing.

According to a preferred embodiment, the first bearing and the third bearing together form a combined axial and radial bearing which serves to receive axial and radial forces. The combined axial and radial bearing preferably comprises an axial bearing and a radial bearing.

According to a preferred embodiment, the first bearing and the third bearing are disposed between the housing and the rotor and the second bearing is disposed on the rotor. The first and third bearing are preferably a combined axial and radial bearing. The axial offset of the first and second permanent magnet is hereby set such that the housing is pressed in the direction of the rotor and/or the rotor is pressed in the direction of the housing.

According to a preferred embodiment, the combined axial and radial bearing is a solid-body bearing, which, in the rotor, preferably comprises a ball which rotates in a cone provided on the housing, thus allowing both radial and axial forces to be received. The material of the ball and/or the cone preferably comprises or consists of monocrystalline corundum or sapphire. These materials are ideal because of their high wear resistance.

According to a preferred embodiment, the first bearing and the third bearing are disposed on the rotor and the second bearing is disposed between the housing and the rotor.

The first and third bearing are preferably mounted on an end of the rotor facing away from the housing. The first and third bearing are mounted between the end of the rotor facing away from the housing and an affixed component, whereby the affixed component is preferably permanently connected to the housing.

The first and third bearing preferably form a combined axial and radial bearing. According to a preferred embodiment, the combined axial and radial bearing is a solid-body bearing, which preferably comprises a ball disposed in the rotor, which rotates in a cone attached to the affixed component, by means of which both radial and axial forces can be received. The material of the ball and/or the cone preferably comprises or consists of monocrystalline corundum or sapphire.

According to a preferred embodiment, the first and the third bearing are realized by two different structural elements. For example, the axial bearing function can be realized by the contact of a ball or other preferably convex surface disposed in the rotor with a preferably flat plate mounted on the housing. The radial bearing function can, for example, be realized by a hydrodynamic sliding bearing on the periphery of the rotor, preferably on the hollow-cylindrical part of the rotor at the level of the second permanent magnet.

The second or third bearing is preferably a hydrodynamic sliding bearing. The second or third bearing is preferably disposed on the periphery of the rotor.

According to a preferred embodiment, the housing is a motor housing which, in its interior, comprises a rotatably mounted shaft and the first permanent magnet disposed on said shaft. It is further preferred that a motor which drives the shaft is disposed in the motor housing. It is further preferred that the motor is completely sealed off from the environment by the motor housing, so that liquid cannot enter the motor housing and the motor, and substances from the interior of the motor also cannot leak into the surrounding liquid.

According to a preferred embodiment, both the first permanent magnet and the second permanent magnet respectively comprise at least one pole pair. The first permanent magnet preferably comprises the same number of pole pairs as the second permanent magnet. It is further preferred that the number of pole pairs is greater than two. This can advantageously ensure that the transmittable torque can be increased.

According to a preferred embodiment, the axial force originating from the coupling, i.e. a magnetic force, which results from the offset of the first and second permanent magnet and acts on the rotor, is selected to be greater than the hydraulic force. In this case, the hydraulic force is a reaction force which acts on the rotor and counteracts the direction of flow. This feature advantageously ensures that, at the first bearing, the rotor is pressed in the direction of the housing, so that the rotor and the housing do not lose contact at the first bearing. The magnetic axial force preferably overcompensates the flow axial force by a safety factor.

According to another preferred embodiment, the axial force originating from the coupling is selected to be smaller than the hydraulic force. The first bearing is only in contact when the system is at a standstill. This means that the hydraulic force on the rotor is at the nominal operating point. In this case, the axial position during operation is entirely determined by the balance between the magnetic force and the hydraulic force.

According to a preferred embodiment, both the first permanent magnet and the second permanent magnet respectively comprise at least two axial segments.

By partitioning the radial coupling, which is realized by the first and second permanent magnet, into two or more segments in axial direction with a simultaneous axial offset of the segments relative to one another, the axial force can advantageously be increased. With comparable dimensions, overall length and outer diameter, the transmittable torque decreases, which can, however, be compensated by an axial elongation of the radial coupling or an increase in the number of pole pairs. Thus, both the torque and the axial force can be adjusted via the number of pole pairs, the exterior dimensions and the partitioning with distances between the segments. The number of segments and the distance between the segments determines the amount of axial force.

This measure can be taken, for example, if the magnetic axial force is insufficient to reliably compensate the flow force.

The number of segments of the first permanent magnet is preferably exactly the same as the number of segments of the second permanent magnet. This simplifies production and increases the symmetry of the device.

The first permanent magnet preferably has the same overall axial length as the second permanent magnet. The overall axial length is the sum of all segments and all spacers. It is hereby assumed that there is no gap between a segment and a spacer or another segment.

According to a preferred embodiment, one spacer is respectively disposed between adjacent segments of the first permanent magnet and/or the second permanent magnet. This can advantageously ensure that the two adjacent segments of a permanent magnet are spaced apart from one another by a predetermined axial distance. This also makes it possible to realize an axial preload, for example in order to be able to produce a defined axial force for a bearing function.

According to a preferred embodiment, at least one spacer comprises or consists of plastic, aluminum, titanium or another non-magnetic material. This has the advantage that the material of the spacer has little or no influence on the magnetic field, since said material is non-ferromagnetic.

According to a preferred embodiment, the second permanent magnet comprises a device for magnetic return. This device is preferably disposed on the outer side of the second permanent magnet. In addition to advantages in terms of production technology, this has the advantage that the torque of the coupling is increased, because fewer stray fields are lost.

According to a preferred embodiment, the first permanent magnet and/or the second permanent magnet has a radial, parallel or diametrical magnetization. These are common types of magnetization, which the person skilled in the art can adapt to the given circumstances of each individual case.

According to a preferred embodiment, the first permanent magnet and/or the second permanent magnet comprises a permanent magnet which comprises or is a Halbach array, i.e. which in particular has the magnet configuration of a Halbach array.

In the present case, a permanent magnet having the magnet configuration of a Halbach array is a permanent magnet in which the magnetic flux density is low on one side, the so-called weak side, because the magnetic flux is essentially canceled there, and high on another side, the so-called strong side, because the magnetic flux is amplified there.

In the present case, a Halbach array is an arrangement of magnets as they are described at the link https://en.wikipedia.org/wiki/Halbach_array, to which reference is hereby made and the disclosure of which is fully incorporated into the description of this invention.

The magnet configuration of a Halbach array can be formed by permanent-magnetic segments which are assembled and the magnetization direction of which relative to one another is tilted 90° with respect to a preferred direction, e.g. with respect to the direction of a longitudinal axis of the arrangement. It is thus possible to achieve a side-dependent flux amplification. For further information, please also refer to the relevant technical literature concerning Halbach arrays.

The first permanent magnet and/or the second permanent magnet is or are preferably a permanent magnet which comprises or is a Halbach array. This feature advantageously ensures that the magnetic flux can be concentrated on one side of the Halbach array (strong side). This is particularly advantageous in the case of the second permanent magnet, which is disposed on the outside, whereby the strong side of the Halbach array of the second breakdown magnet [sic] is directed toward the first permanent magnet.

The first permanent magnet and the second permanent magnet are magnetized such that a rotation of the first permanent magnet sets the second permanent magnet in rotation and vice versa. This characteristic is necessary to be able to transmit torque from the one permanent magnet to the other permanent magnet without contact.

Preferably, the first permanent magnet and the second permanent magnet together form a magnetic coupling which, due to the preferably radially directed magnetic field lines, is preferably a radial magnetic coupling.

According to a preferred embodiment, an axial force of the rotor bearing system can be freely adjusted by varying at least one of the following list. The list includes: a pole pair number of the first permanent magnet and the second permanent magnet; the dimensions of the segments of the first permanent magnet; the dimensions of the segments of the second permanent magnet; distances between adjacent segments of the first permanent magnet and the second permanent magnet;

distances between adjacent segments of the magnetic return; axial lengths of spacers between segments of the first permanent magnet and the second permanent magnet and segments of the magnetic return; a magnetization of the first permanent magnet; a magnetization of the second permanent magnet; a flow force which acts on the rotor during proper use; and an offset of the first permanent magnet relative to the second permanent magnet.

The person skilled in the art knows that the variables mentioned in the list influence the axial force. By varying at least one of the values in the list, preferably several values in the list, the axial force can be freely adjusted within predetermined limits. This can advantageously ensure that the axial force can appropriately be adapted to the given circumstances of each individual case.

The rotor preferably comprises at least one bore or at least one hole, preferably bores or holes. This advantageously ensures that the liquid transported by the rotor can flow into a space or gap between the rotor and the housing. This enables heat, produced for example by friction or by eddy currents in a possibly metal housing, to be dissipated. Furthermore, the continuous flow of the medium ideally prevents deposits of solid particles of the medium in the region of the gap and the bearing.

### BRIEF DESCRIPTION OF THE DRAWINGS

Advantageous design examples of the invention are shown in the drawings and are explained in more detail in the following description.

FIG. 1 shows a rotor bearing system according to an embodiment of the invention.

FIG. 2 shows a sectional view through an embodiment of the rotor bearing system according to the invention at a position in which the first permanent magnet, which is mounted in the housing, and the second permanent magnet, which is disposed in the rotor, overlap.

FIGS. 3 and 4 respectively show a rotor bearing system according to further embodiments of the invention.

### DETAILED DESCRIPTION

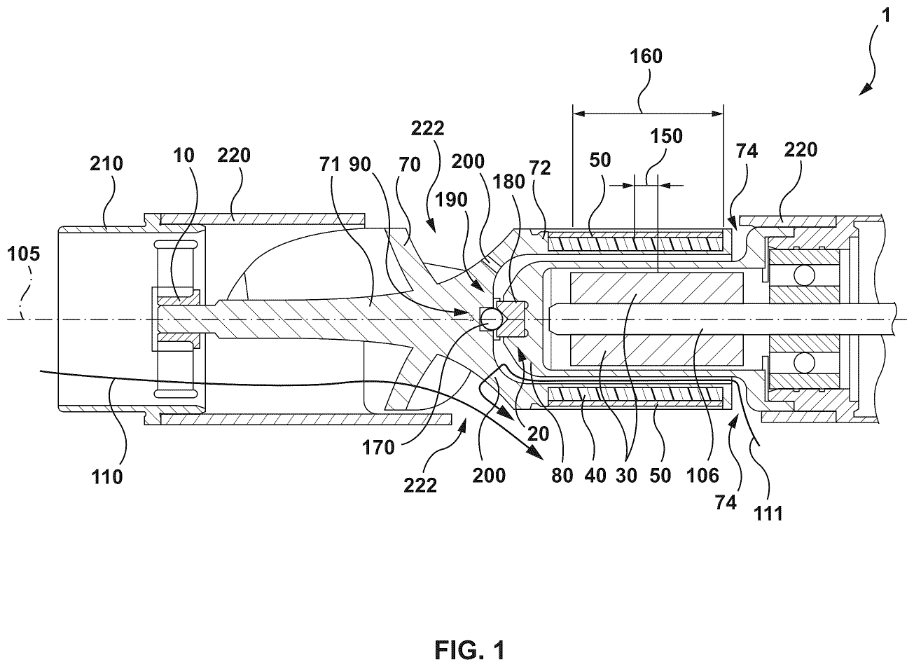

FIG. 1 shows a rotor bearing system 1 with contactless torque transmission and a radial and axial bearing of a rotor, which is designed in the form of a pump for cardiovascular support (VAD), for example.

The rotor bearing system 1 comprises a housing 80, here a motor housing, in which a first cylindrical permanent magnet 30 is seated and mounted on a shaft 106 driven by a not depicted motor such that it can rotate about a first axis 105. The housing 80 has an outer diameter of 3.5 mm.

The rotor bearing system 1 further comprises a rotor 70 for conveying a liquid, wherein the rotor 70 comprises a second hollow-cylindrical permanent magnet 40, which is likewise mounted such that it can rotate about the first axis 105. The second hollow-cylindrical permanent magnet 40 is mounted in a hollow-cylindrical part 72 of the rotor 70.

The second hollow-cylindrical permanent magnet 40 comprises a magnetic return 50 on its outer side.

The first permanent magnet 30 has an outer diameter of 3 mm, a magnet height of 1 mm and a length of 5 mm. The second permanent magnet 40 has an outer diameter of 5 mm, a magnet height of 0.5 mm and a length of 5 mm. The rotor 70 has an outer diameter of 5.3 mm and a length of 15 mm.

The rotor 70 is designed as an impeller, which converts the mechanical power transmitted by the coupling into hydraulic power for supporting a blood flow against a blood pressure.

The rotor 70 further comprises a conical or tapered part 71, which adjoins the hollow-cylindrical part 72. The outer circumference of the base of the conical part 71 is connected to the annular opening at an axial end of the hollow-cylindrical part 72.

The first permanent magnet 30 and the second permanent magnet 40 overlap axially at least partially in the axial region identified with the reference sign 160.

The first permanent magnet 30 is hereby disposed axially offset relative to the second permanent magnet 40. The centers of the first permanent magnet 30 and the second permanent magnet 40 are marked by vertical lines and the axial offset 150 is drawn in between these two dashed lines.

As a result of the axial offset 150, the second permanent magnet 40 experiences a force which, in FIG. 1, is directed toward the right, so that a ball 170 disposed in the rotor 70 is pressed onto a cone 180 mounted in the housing 80, so that a first bearing 20 and a third bearing 90, which here form a combined axial and radial bearing 190, is kept in contact. During proper use, the ball 170 rotates in the cone 180, as a result of which both radial and axial forces can be received. The combined axial and radial bearing 190 here is a solid-body bearing. The ball 170 is disposed in the conical part 71. The axial and radial bearing function is achieved by combining the two elements ball 170 and cone 180.

The ball 170 has a diameter of 0.5 mm. The cone 180 has a diameter of 1 mm, a height of 0.8 mm and a cone angle of 90°.

The axial bearing function of the combined bearing 190 functions as the first bearing and is used for the relative axial positioning of the rotor 70 and the housing 80 or the shaft 106 relative to one another, and also for receiving an axial force resulting from the arrangement of the first permanent magnet 30 and the second permanent magnet 40.

The axial force in the rotor bearing system 1 can furthermore be freely adjusted, as a result of which the acting forces can be optimally adjusted.

In the overlap region 160 and in the region between the overlap region 160 and the rotor 70, the housing 80, which comprises the first permanent magnet 30, is surrounded by the rotor 70, in particular by the interior of the hollow-cylindrical part 72 of the rotor 70. A hollow-cylindrical channel 74 through which the liquid can flow is thus formed between the housing 80 and the rotor 70. In order to allow liquid to flow continuously from outside the conical part 71 of the rotor 70 into the channel 74, bores 200 are drilled into the rotor 70, preferably in the conical part 71 of the rotor 70, or at a transition from the conical part 71 to the hollow-cylindrical part 72 of the rotor 70. The flow direction of the liquid is indicated by arrow 110. Arrow 111 indicates a direction of flow of liquid through the channel 74.

A second bearing 10, which is designed as a radial, hydrodynamic and blood-lubricated sliding bearing, is mounted on the end of the conical part 71 of the rotor 70 facing away from the housing 80. The second bearing 10 is used to receive radial forces and to position the axis of rotation of the second permanent magnet 40, which is disposed in the rotor 70. The second bearing 10 is disposed between the rotor 70 and an insert 210 which is mounted, in particular clamped or press-fitted, on a second housing 220 in an annular end, which is in turn mounted on the housing 80. The second housing 220 forms an outer skin of the rotor bearing system 1, whereby numerous outlet windows 222 are present in the second housing 220, which can also be referred to as an impeller housing. The insert 210 is preferably a spider bearing that can be glued, welded or press-fitted into the second housing 220. The spider bearing 210 has an outer diameter of 6 mm and a length of 3 mm. The second housing 220 has an outer diameter of 6 mm, a length of 18 mm and a wall thickness of 0.25 mm.

The bearing 10 has a diameter of 1 mm and a length of 1 mm.

As a result of the axial offset 150 between the first permanent magnet 30 and the second permanent magnet 40 established by the design, in the design example of FIG. 1 a defined axial force acts on the rotor 70 in the direction of the motor; i.e. from left to right in the design example of FIG. 1. This force is counteracted by a hydraulic force on the rotor 70, i.e. from right to left in the design example of FIG. 1. In the present case, the axial force originating from the coupling of the first permanent magnet 30 and the second permanent magnet 40 is selected to be slightly greater than the hydraulic force. On the one hand, this ensures that the rotor 70 is always in a defined axial position and, on the other hand, that the combined axial and radial bearing 190 is not unnecessarily loaded. Consequently, friction and wear are kept low. To optimize the friction and wear behavior, the cone angle of the cone 180 can also be increased, whereby a sufficient radial load-bearing capacity has to be ensured.

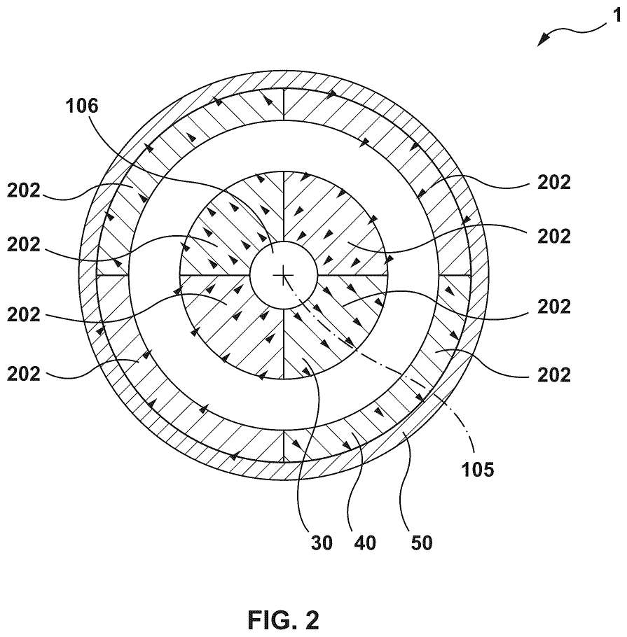

FIG. 2 shows a sectional view of the rotor bearing system 1 at a position in which the first permanent magnet 30, which is mounted in the housing 80, and the second permanent magnet 40, which is disposed in the hollow-cylindrical part 72 of the rotor 70, overlap axially. It can be seen that the first permanent magnet 30 is seated on the shaft 106 which is driven by the motor and is mounted such that it can rotate about the first axis 105. It can also be seen that the second permanent magnet 40 is likewise mounted such that it can rotate about the first axis 105. Both the first permanent magnet 30 and the second permanent magnet 40 comprise two pole pairs, i.e. four poles 202 each, which are respectively radially magnetized as indicated by small arrows.

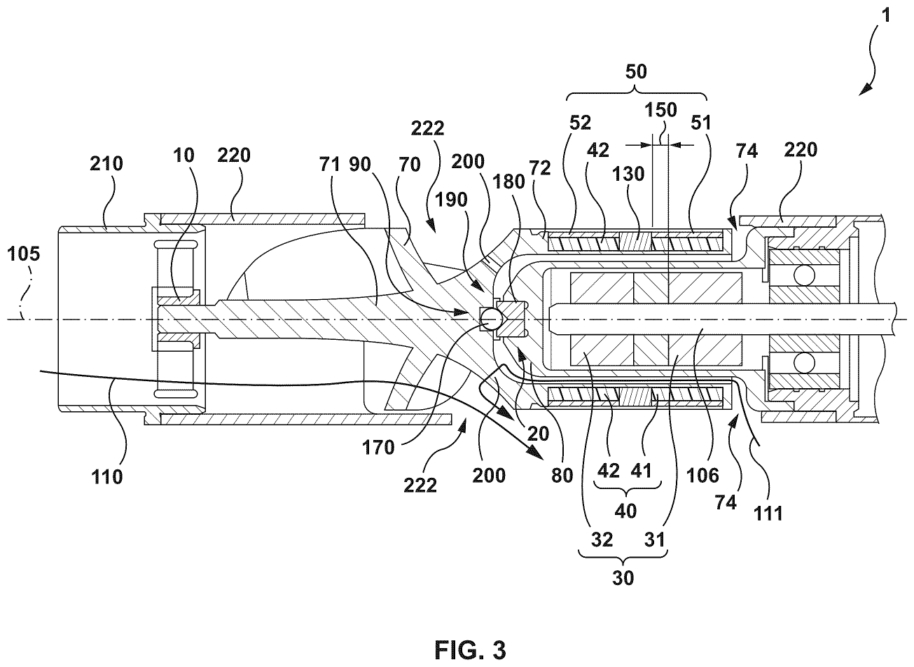

FIG. 3 shows a rotor bearing system 1 in a similar embodiment as the rotor bearing system 1 of FIG. 1. The present embodiment differs from the embodiment of FIG. 1 in that the first permanent magnet 30, the second permanent magnet 40 and the magnetic return 50 are all divided into two axial segments.

The first permanent magnet 30 comprises the segments 31 and 32, the second permanent magnet 40 comprises the segments 41 and 42, and the magnetic return 50 comprises the segments 51 and 52. The segments 31, 41 and 51 are disposed on the motor side and the segments 32, 42 and 52 are disposed on the side facing the rotor 70.

A hollow-cylindrical and non-magnetic spacer 130, which is likewise mounted on the shaft 106, is disposed between the segments 31 and 32. A further hollow-cylindrical and non-magnetic spacer 130 is disposed between the segments 41 and 51 on the one side and the segments 42 and 52 on the other.

The segmentation in combination with the offset 150 between the two axial halves leads to an increase in the magnetic axial force while, at the same time, the transmittable torque decreases. This measure is taken in the present case because the magnetic axial force is insufficient to reliably compensate the flow force.

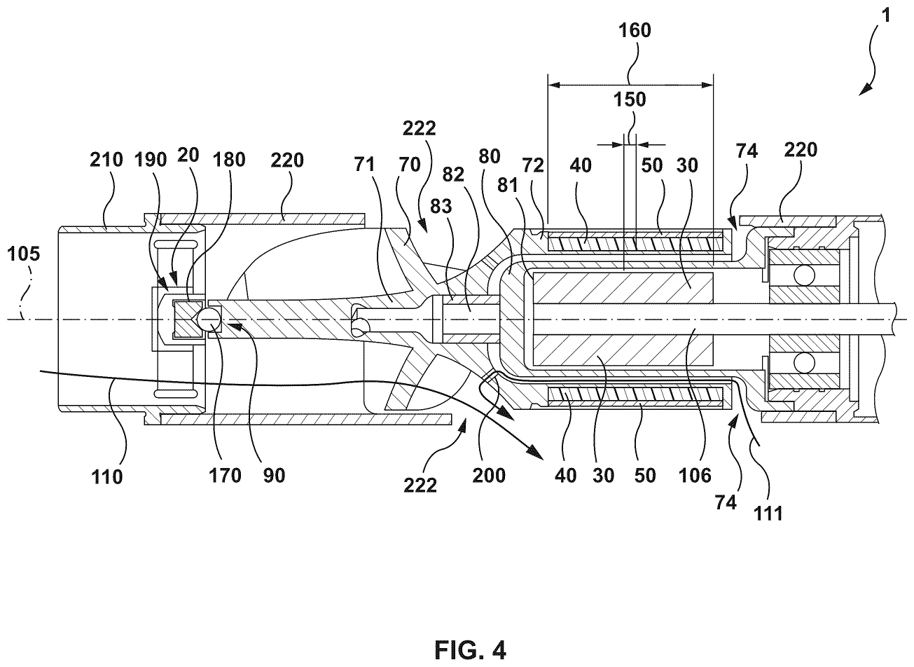

FIG. 4 shows a rotor bearing system 1 in a similar embodiment as the rotor bearing system 1 of FIGS. 1 and 3. The present embodiment differs from the embodiment of FIG. 1 in that, on the one hand, the position of the second bearing 10 is interchanged with the position of the first bearing 20 and the third bearing 90 and, on the other hand, the axial offset 150 between the first permanent magnet 30 and the second permanent magnet 40 points in the opposite direction as in the embodiment of FIG. 1. In the embodiment of FIG. 4, the axial offset 150 is 1 mm.

The first permanent magnet 30 and the second permanent magnet 40 overlap axially at least partially in the axial region identified with the reference sign 160. The first permanent magnet 30 is hereby disposed axially offset relative to the second permanent magnet 40. The centers of the first permanent magnet 30 and the second permanent magnet 40 are marked by vertical dashed lines and the axial offset 150 is drawn in between these two vertical dashed lines. In contrast to the embodiment of FIG. 1, viewed from the housing 80, the first permanent magnet 30 is axially offset relative to the second permanent magnet 40 in the direction of the rotor 70. Between the first permanent magnet 30 and the second permanent magnet 40 in the design example of FIG. 4, there is therefore a defined axial force which acts on the rotor 70 and is directed from the housing 80 toward the rotor 70 along the axis 105; i.e. from right to left in the design example of FIG. 4. A hydraulic force acts on the rotor 70 in the same direction, i.e. likewise from right to left in the design example of FIG. 4.

The advantage of this arrangement is that both the magnetic and the hydraulic axial force on the rotor 70 point in the same direction, namely upstream, as a result of which the rotor 70 is continuously pressed into the combined axial and radial bearing 190.

The first bearing 20 and the third bearing 90 here too form a combined axial and radial bearing 190, which is mounted on the end of the conical part 71 of the rotor 70 facing away from the housing 80. The combined bearing 190 is disposed between the rotor 70 and an insert 210 which is mounted, in particular clamped, on a second housing 220 in an annular end, which is in turn mounted on the housing 80. A ball 170, which is disposed on the end of the conical part 71 of the rotor 70 facing away from the housing, is hereby pressed onto a cone 180 mounted on the insert 210.

The second bearing 10, which is configured as radial, hydrodynamic sliding bearing, is used to receive radial forces and to position the axis of rotation of the second permanent magnet 40, which is disposed in the rotor 70. The second bearing 10 is disposed between the housing 80 and the rotor 70. In contrast to the embodiment of FIG. 1, behind a wall 81 facing toward the rotor 70, the housing 80 of the embodiment of FIG. 4 comprises a cylindrical journal 82 which continues as an extension of the shaft 106 in the direction of the rotor 70. The journal 82 is surrounded by a bearing shell 83 of the radial sliding bearing which forms the second bearing 10.

In summary, the following preferred features of the invention should in particular be noted:

The invention relates to a rotor bearing system 1. Said system comprises a housing 80 in which a first permanent magnet 30 is mounted such that it can rotate about a first axis 105. A rotor 70 for conveying a liquid comprises a second hollow-cylindrical permanent magnet 40, which is mounted such that it can rotate about a second axis. The first permanent magnet 30 and the second permanent magnet 40 overlap axially at least partially, wherein the first permanent magnet 30 is disposed offset relative to the second permanent magnet 40. In the axial overlap region 160 of the first permanent magnet 30 and the second permanent magnet 40, the housing 80 is positioned between the two permanent magnets 30, 40. A first bearing 20 is configured for the relative axial positioning of the rotor 70 and the housing 80 with respect to one another and for receiving an axial force resulting from the arrangement of the first permanent magnet 30 and the second permanent magnet 40, and a second bearing 10 and a third bearing 90 are configured for receiving radial forces and for positioning the axis of rotation of the second permanent magnet 40.

1. (canceled)

2. A rotor bearing system comprising:
a first permanent magnet configured to rotate about an axis;
a rotor configured to convey a liquid, wherein the rotor comprises a second permanent magnet, wherein the second permanent magnet comprises a hollow permanent magnet and is configured to rotate about the axis;
wherein the first permanent magnet and the second permanent magnet are axially offset and arranged to produce an axial force; and
a bearing configured to provide a relative axial positioning of the rotor and the first permanent magnet with respect to one another and configured to receive the axial force resulting from an arrangement of the first permanent magnet and the second permanent magnet.

3. The rotor bearing system of claim 2, wherein the first permanent magnet is mounted within a housing.

4. The rotor bearing system of claim 2, wherein the second permanent magnet is cylindrical.

5. The rotor bearing system of claim 2, wherein the first permanent magnet and the second permanent magnet at least partially overlap axially.

6. The rotor bearing system of claim 5, wherein a housing is positioned between the first permanent magnet and the second permanent magnet in an axial overlap region of the first permanent magnet and the second permanent magnet.

7. The rotor bearing system of claim 2, further comprising a second bearing, wherein the bearing and the second bearing are configured to receive radial forces and configured to position the axis of rotation of the second permanent magnet.

8. The rotor bearing system of claim 7, wherein the bearing is disposed between the first permanent magnet and the rotor and the second bearing is disposed on the rotor.

9. The rotor bearing system of claim 2, wherein each of the first permanent magnet and the second permanent magnet comprise at least one pair of poles, wherein the first permanent magnet and the second permanent magnet comprise an equal number of pairs of poles.

10. The rotor bearing system of claim 2, wherein the axial force resulting from the arrangement of the first permanent magnet and the second permanent magnet is configured to be greater than a hydraulic force acting on the rotor.

11. A heart support system comprising:
a rotor bearing system comprising:
a first permanent magnet configured to rotate about an axis;
a rotor configured to convey a liquid, wherein the rotor comprises a second permanent magnet, wherein the second permanent magnet comprises a hollow permanent magnet and is configured to rotate about the axis;
wherein the first permanent magnet and the second permanent magnet are axially offset and arranged to produce an axial force; and
a bearing configured to provide a relative axial positioning of the rotor and the first permanent magnet with respect to one another and configured to receive the axial force resulting from an arrangement of the first permanent magnet and the second permanent magnet.

12. The heart support system of claim 11, wherein the first permanent magnet is mounted within a housing.

13. The heart support system of claim 11, wherein the second permanent magnet is cylindrical.

14. The heart support system of claim 11, wherein the first permanent magnet and the second permanent magnet at least partially overlap axially.

15. The heart support system of claim 14, wherein a housing is positioned between the first permanent magnet and the second permanent magnet in an axial overlap region of the first permanent magnet and the second permanent magnet.

16. The heart support system of claim 11, further comprising a second bearing, wherein the bearing and the second bearing are configured to receive radial forces and configured to position the axis of rotation of the second permanent magnet.

17. The heart support system of claim 16, wherein the bearing is disposed between the first permanent magnet and the rotor and the second bearing is disposed on the rotor.

18. The heart support system of claim 11, wherein each of the first permanent magnet and the second permanent magnet comprise at least one pair of poles, wherein the first permanent magnet and the second permanent magnet comprise an equal number of pairs of poles.

19. The heart support system of claim 11, wherein the axial force resulting from the arrangement of the first permanent magnet and the second permanent magnet is configured to be greater than a hydraulic force acting on the rotor.
